(19) United States
(12) Patent Application Publication
(10) Pub. No.: US 2009/0130166 A1
(43) Pub. Date: May 21, 2009

(54) SYSTEM FOR PROVIDING IMPLANT COMPATIBILITY WITH RECIPIENT

(75) Inventor: Nicanor I. Moldovan, Dublin, OH (US)

Correspondence Address: CALFEE HALTER & GRISWOLD, LLP, 800 SUPERIOR AVENUE, SUITE 1400, CLEVELAND, OH, 44114, US

(73) Assignee: THE OHIO STATE UNIVERSITY, Columbus, OH (US)

(21) Appl. No.: 12/233,581

(22) Filed: Sep. 18, 2008

Related U.S. Application Data
(63) Continuation of application No. 11/085,445, filed on Mar. 21, 2005, now abandoned.
(60) Provisional application No. 60/554,592, filed on Mar. 19, 2004.

(51) Int. Cl. A61F 2/04; A61K 35/12

(52) U.S. Cl. 424/423; 424/93.7

(57) ABSTRACT

A system for providing or enhancing compatibility between an implant and a recipient. The system includes a recipient, typically a living biological organism, that further includes a target region; an implant having a first attraction means; and a plurality of cells. The cells are compatible with the target region and further include a second attraction means responsive to the first attraction means. The interaction between the first and second attraction means attaches the cells to the implant.

### CROSS-REFERENCE TO RELATED APPLICATIONS

This patent application is a continuation of U.S. patent application Ser. No. 11/085,445 filed on Mar. 21, 2005, which claims priority to U.S. Provisional Patent Application Ser. No. 60/554,592 filed on Mar. 19, 2004, the disclosures of which are incorporated by reference herein.

### TECHNICAL FIELD

This invention relates in general to systems and methods involving the implantation of an implant into a recipient organism, and in particular to a system for improving the attachment of cells to the surface of an implant for the purpose of increasing or enhancing the compatibility of the implant with the recipient organism.

### BACKGROUND

Implants are known to be useful for a variety of purposes such as, for example, controlled-release drug delivery, tissue or bone engineering, and cardiovascular applications. When in use, such implants, which may be manufactured from a variety of materials, may cause undesirable side affects or create other problems following implantation into the body of a living organism. Implantation is by its nature an invasive procedure and access to the tissue is created during implantation. The produced wound and its consequent healing limit integration of the implant in the organ. Recipient immune system rejection, excessive scarring, and restenosis are examples of problems frequently encountered with the use of such devices.

Coating the exterior of an implant device with certain cell types has been attempted for the purpose of improving implant biocompatibility and performance. This approach may include the use of gravity for encouraging cells to settle on the flat or planar surfaces of an device prior to implantation. This method of implant coating is only marginally effective because many implant devices include multiple contoured surfaces. Uniform cell coverage is difficult to attain because cells will not typically adhere to the contoured surface of the implant. Other known methods for encouraging cell attachment such as centrifugation or the use of gel constructs are usually cumbersome, time-consuming, and/or may limit the effectiveness or function of the implant, especially in situations where the implant is a controlled-release drug delivery device or biosensor. Thus, there is a need for implant devices to exhibit improved biocompatibility with the recipient organism, and there is a need for a system that improves cell attachment to implant surfaces for providing improved biocompatibility.

### SUMMARY

Deficiencies in and of the prior art are overcome by the present invention, the exemplary embodiment of which provides a system for providing or enhancing compatibility between an implant and a recipient. An exemplary embodiment of this system includes a recipient, typically a living biological organism, that further includes a target region; an implant having a first attraction means; and a plurality of cells. The cells are compatible with the target region and further include a second attraction means responsive to the first attraction means. The interaction between the first and second attraction means attaches the cells to the implant. The attraction means may be magnetic and the implant may be a drug delivery device, pacemaker, stent, biosensor, orthopedic device, or other article, item, or device. The cells may be stem, progenitor, or mature cells and the implant may include plastic, silicon, metal, or a combination thereof. Other cell types and materials are possible.

Another embodiment of this invention provides a method for increasing the bioavailability of a biologically active agent or composition placed in or on an implant device. An exemplary embodiment of this method includes the steps of placing a biologically active agent in or on an implant, wherein the implant further comprises multiple surfaces and a magnet or magnetic source in temporary or permanent communication with at least one of the multiple surfaces of the implant. The implant is then seeded with vascular precursor cells or other cells that have been magnetically labeled. The magnetic attraction between the magnet and the magnetically labeled cells attaches the cells to the surface of the implant opposite the magnet. The implant is then placed, i.e., implanted, into a target region and the vascular precursor cells or other cells at the implant surface differentiate and/or proliferate to substantially vascularize the implant and, in some cases, the tissue surrounding the implant. The agent is released from the implant and enters the vasculature, i.e., the vessel network surrounding the implant, thereby increasing the bioavailability of the agent. In addition to magnetic attraction between the cells and implant, other attraction means may be employed.

Additional features and aspects of the present invention will become apparent to those of ordinary skill in the art upon reading and understanding the following detailed description of the exemplary embodiments. As will be appreciated, further embodiments of the invention are possible without departing from the scope and spirit of the invention. Accordingly, the drawings and associated descriptions are to be regarded as illustrative and not restrictive in nature.

### BRIEF DESCRIPTION OF THE DRAWINGS

The accompanying drawings, which are incorporated into and form a part of the specification, schematically illustrate one or more exemplary embodiments of the invention and, together with the general description given above and detailed description of the preferred embodiments given below, serve to explain the principles of the invention.

FIGS. 1A-B are schematic representations of the biocompatibility system and implant.

FIG. 2A-D are photographs of labeled and non-labeled cells attached or not attached to a magnetic or non-magnetic implant.

FIG. 3A-B are photographs of labeled cells showing alignment of magnetic beads within the cells and alignment of the cells themselves using an external magnetic force.

### DETAILED DESCRIPTION

This invention relates to a system for increasing the likelihood that an implant that is placed, i.e., implanted, within a target region in the body of a living biological organism (e.g., human or animal) will be accepted or at least tolerated by the organism and that excessive scarring, immune system rejection, restenosis, and/or other possible negative outcomes or effects will be reduced or eliminated. Using the methods of this invention, cell coverage of the surface is relatively rapid and substantially uniform regardless of the geometry or shape (e.g. flat, curved, etc.) of the implant.

In the exemplary embodiments of this invention, at least one surface of a implant is coated, covered, or seeded with “precursor” cells, i.e., stem cells, progenitor cells, or mature cells of one or more specific cell types for the purpose of minimizing negative effects that the implant may have on the recipient following implantation. This seeding may be done entirely ex vivo, although other preparation methods are possible. The implant or at least one surface of the implant includes a first attraction force or means and the cells are labeled with or otherwise include a second attraction force or means responsive to the first attraction force or means. The interaction of the attraction forces or means causes the cells to attach or adhere to the surface of the implant. Preferably, the entire implant is uniformly coated with cells prior to or shortly after implantation. Presumably, uniformly covering the implant device with one or more of these cells types will decrease the formation of fibrous or scar tissue near or around the implant and may also increase vascularization of the tissue surrounding the device. Providing precursor cells that have a phenotype similar to that of the host or recipient tissue will presumably limit the amplitude of foreign body immune reaction and will speed recovery following implantation. If the implant device is functioning as a controlled-release device, stimulating and/or differentiating growth factors may be included in the formulation being released to enhance the proliferation and/or differentiation of the precursor cells following implantation. Rejuvenation of local tissue cells may also be possible through the use of certain types of progenitor cells attached to the implant.

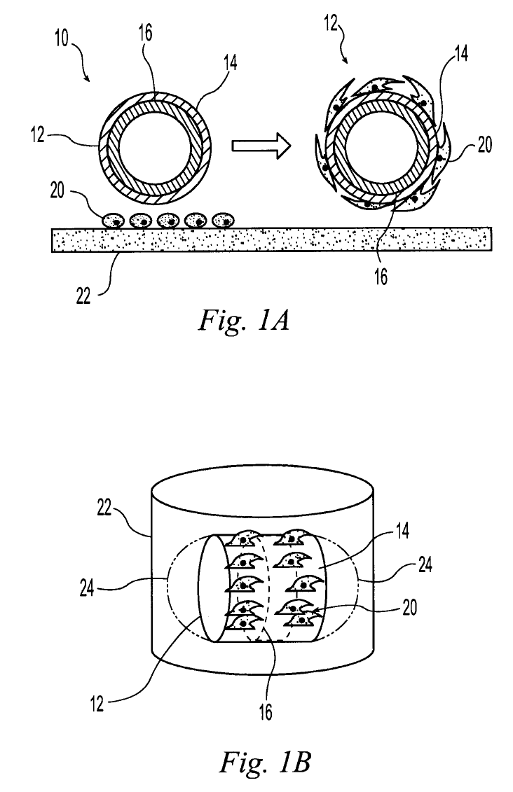

With reference to FIG. 1A, one embodiment of the present invention provides a system 10 for providing compatibility between an implant 12 and a target region. This system includes (a) a biological target region, wherein the target region comprises predetermined cellular or tissue characteristics (e.g., bone or cardiovascular tissue); (b) an implant 12 for placement within the target region, wherein the implant further comprises: (i) a substrate 14, wherein the substrate further comprises multiple, i.e., at least two, surfaces; (ii) at least one magnet 16 in communication with at least one surface of substrate 16; (iii) a plurality of magnetically labeled cells 20, wherein the cell type may be determined by the characteristics of the target region or by other factors. The magnetic attraction between the magnet and the magnetically labeled cells attaches the magnetically labeled cells to the surface of the substrate opposite the magnet. This attachment is illustrated in FIG. 1A from left to right as the cells 20 move from reservoir 22 to the surface of implant 12. Although shown on the inside of substrate 14 in the Figures, magnet 16 may be on the outside of the substrate or embedded within the substrate. Multiple magnets of varying strengths may be used and multiple cell types may be attached to the surface of the implant. Additionally, the magnet may be permanently placed in communication with the substrate or it may be temporarily placed in communication with the substrate, i.e., it may be removed after attachment of the cells to the implant. Alternately, the magnet or source of magnetic force may be incorporated directly into the substrate.

With reference to FIG. 1B, another embodiment of the present invention provides an implant device 12 for implantation within a biological target region. In this figure, implant device 12 is shown within a reservoir 22. Device 12 includes a substrate 14, wherein the substrate further comprises multiple surfaces, some of which may be contoured, i.e., curved; (b) at least one magnet 16 in communication with at least one surface of substrate 14; and (c) a plurality of magnetically labeled cells 20. The magnetic attraction between magnet 16 and magnetically labeled cells 20 attaches the magnetically labeled cells to the surface of the substrate opposite the magnet. Although shown on the inside of substrate 14 in the Figures, magnet 16 may be on the outside of the substrate or embedded within the substrate. Multiple magnets of varying strengths may be used.

A third embodiment of the present invention provides a method for increasing the biocompatibility between an implant and a target region. This method includes the steps of constructing an implant, wherein the implant further comprises: (i) at least two surfaces; and (ii) at least one magnet in communication with at least one surface; and (b) seeding the implant with magnetically labeled cells, wherein the cells are compatible within the target region, and wherein the magnetic attraction between the magnet and the magnetically labeled cells attaches the cells to the surface opposite the magnet; and (c) placing the implant into a target region, wherein the cells proliferate to substantially cover the implant.

A fourth embodiment of this invention provides a method for increasing the bioavailability of a drug or other bioactive agent released from or by an implant. This method includes placing a drug or other composition in or on an implant, wherein the implant further comprises: (i) a substrate having multiple surfaces; and (ii) at least one magnet in communication with at least one surface of the substrate. The implant is then seeded with precursor cells (e.g., human endothelial cells or other vascular precursor cells), wherein the cells are magnetically labeled, and wherein the magnetic attraction between the magnet and the magnetically labeled precursors cells attaches the cells to the surface of the substrate opposite the magnet; and (c) placing the implant into a target region, wherein the precursor cells differentiate and proliferate to substantially vascularize the tissue surrounding the implant, and wherein the drug or bioactive agent is released from the implant and enters the vasculature, i.e., vessel network surrounding the implant, thereby increasing the bioavailability of the drug.

Having generally described this invention, a further understanding can be obtained by reference to certain specific examples detailed below, which are provided for purposes of illustration only and are not intended to be all inclusive or limiting unless otherwise specified. The procedures described below provide an example of how the methods of the present invention are implemented to coat an implant with human endothelial cells. In this Example, cultured cells were seeded onto a cylindrical plastic substrate containing a magnet. As shown in the Figures, the labeled cells attached to the surface of the substrate containing the magnet and did not attach to the surface of the same substrate without the magnet.

### EXAMPLE

### Preparation of Magnetically Labeled Cells

In this example, cells for use with the implant were labeled using the commercially available Dynabeads labeling system purchased from Dynal Biotech (Lake Success, N.Y.). Dynabeads CD31 are uniform, superparamagnetic polystyrene beads (4.5 μm in diameter) coated with a mouse IgG1 monoclonal antibody specific for the CD31 cell surface antigen PECAM-1 (platelet endothelial cell adhesion molecule-1). The following description is adapted from the protocol that accompanies the purchased product. Step 1: Washing the Beads. The Dynabeads CD31 were resuspended, and then transferred to a tube to which 1 ml of a first buffer solution was added. The tube was placed within a magnet and the supernatant was discarded. The tube was removed from the magnet and the beads were resuspended in the same volume of the first buffer solution as was initially used. Step 2: Preparation of Single Cell Suspension: a single cell suspension of Human Umbilical Cord Vein Endothelial Cells (HUVEC) (Clonetics/Cambrex, MD), was prepared according to published protocols (see: Jackson et al., Cell Sci. 96: 257-262 (1990); Jaffe et al., Clin. Invest. 52: 2745-2756 (1973); and Mutin et al., Tissue Antigens 50:449-458 (1997)). Step 3: Positive Isolation of HUVEC Cells from Suspension. An appropriate volume of CD31 Dynabeads was added to a tube of prepared cell suspension, incubated for 20 minutes (positive isolation) or 30 minutes (depletion) at 2-8° C. with gentle tilting and rotation, and the tube was placed in a magnet for 2 minutes. For depletion, the supernatant is transferred to a new tube for further use; for positive isolation, the supernatant is discarded and the bead-bounds cells are washed 3 times by resuspending in the first buffer solution to the original sample volume, and separating using a magnet.

Regarding the magnetic labeling of cells, it was observed that following the initial labeling step, the cells tend to surround and internalize up to about 10 magnetic beads each. Each time a bead-containing cell divides, the number of beads per cell decreases until the magnetic aspect is rendered ineffective or lost altogether from the cell population. Thus, high density seeding of an implant results in greater retention of the magnetic affect because cell division is inhibited. Low density seeding provides a means by which to quickly reduce or remove the magnetic effect created by labeling cells with magnetic or paramagnetic beads.

### Preparation of Cell-Covered Implant

In this example, a plastic tubular substrate, i.e., implant, was coated with human umbilical vein endothelial cells (HUVECs) (Clonetics/Cambrex, MD) labeled with superparamagnetic CD31 Dynabeads (Dynal Biotech, Lake Success, N.Y.) per the protocol discussed in the preceding paragraph. The polymeric caps of the hollow implant were opened, a magnet was introduced into the hollow space, and the caps were closed. The implant was then rolled or dipped into the cell suspension and the labeled cells were allowed to adhere to the outer surface of the implant for about 30 minutes to 1 hour. During this period of time, the implant was placed in a suitable growth medium at 37° C. These steps were all performed under sterile conditions. Following the cell adherence/adhesion step, the magnet may be removed from the implant or it may be left in place depending on the specific application. Magnetic field strengths may also be varied in situations where resistance to shear due to flow is desirable for encouraging the cells to stay adhered to the substrate. In alternate embodiments, the magnet may be placed on the outside surface of the implant to allow the labeled cells to bind to the inner surface of the implant.

### Analysis of Cell Adhesion

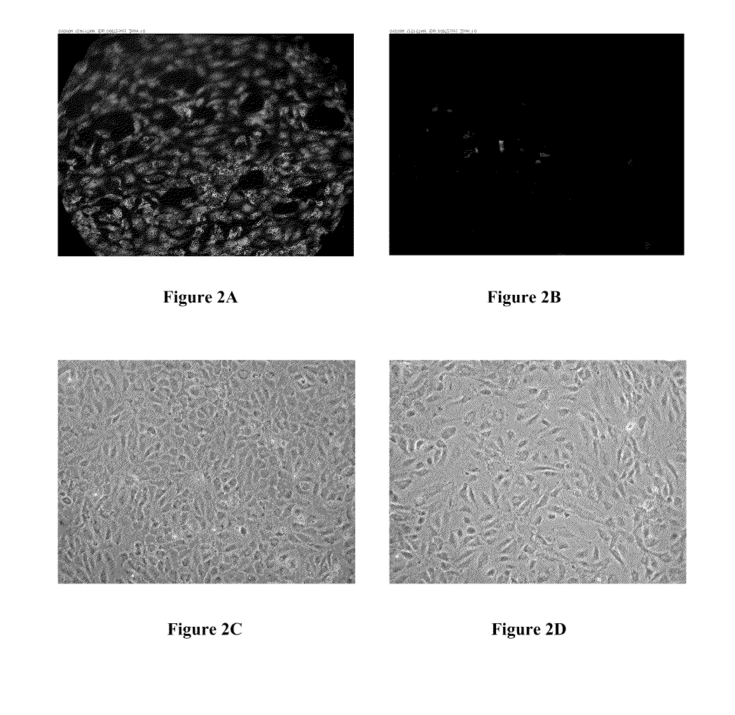

Cells were allowed to grow for 48 hours after which they were stained with cell tracker green (Molecular Probes: Eugene, Oreg.) to facilitate microscopic visualization. The photograph of FIG. 2A shows that the surface of the magnetic implant was sufficiently coated with magnetically labeled endothelial cells using the method of the present invention. FIG. 2A shows the surface of the magnetic implant covered with magnetically labeled human endothelial cells. FIG. 2B shows the lack of labeled cells on the surface of the non-magnetic implant. FIG. 2C shows non-labeled cells not adhering to the surface of the magnetic implant. FIG. 2D shows non-labeled cells not adhering to the surface the non-magnetic implant. All photographs are at 20× magnification.

In the Example, mature endothelial cells were chosen both to demonstrate the effective “seeding” of the implant and because after implantation, endothelial cells proliferate and provide enhanced implant vascularization. As described above, enhanced vascularization provides a vessel network that may increase the bioavailability of the implant's drug content. Multiple cell types may be used simultaneously to cover the implant, including mixtures (or layers) of various cells, including tissue-specific cells (bone, cardiac, etc) with non-specific vascular progenitors, seeded together or sequentially on the implant. Genetically engineered cells may also be used and may provide stimulation of neovascularization in peri-implant regions; limitation of the immune/foreign body reaction, correction of the organ functions, or other functions.

Advantageously, the present invention is compatible with a variety of implants types and materials (e.g. plastic, silicon, titanium) and may be used for multiple therapeutic applications, including: cardiovascular applications (e.g., pacemakers, stents, and vascular prostheses); bone and tissue engineering (e.g., orthopedic: strengthening the interface between a metal implant and bone); mechanical, electrical, or passive subcutaneous implants; implantable drug delivery devices, including controlled-delivery devices; and biosensors. Essentially, this invention may be used in most, if not all, situations where seeding, frosting, or coating the exterior of an implant will (i) increase the implant's compatibility with the recipient's biology or physiology; (ii) increase or enhance the performance and/or function of the implant device or implant system; or optimize the tissue healing and response after implantation. For most applications, the system and device of this invention may be assembled using commercially available materials, thereby reducing costs and adding simplicity to the overall process.

Various types of magnetic beads may be used for labeling the cells used to cover the implants described herein. Such beads may vary in size (e.g., from microns to nanometers) and in the nature of the magnetic material (e.g., magnetic, paramagnetic etc), thereby leading to various different methods of labeling and cell incorporation, final localization within cells, strengths of the magnetic forces acting upon the cells, and methods of detection following implantation in the recipient.

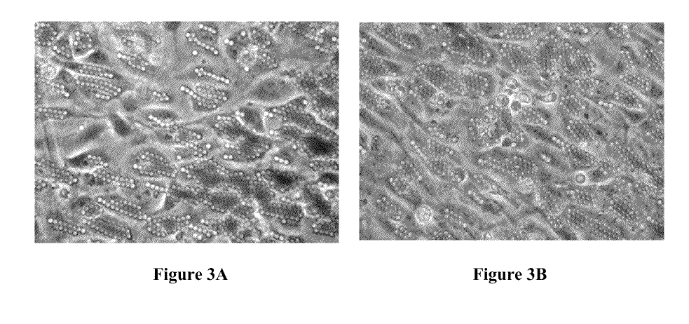

With reference to FIGS. 3A-B, other embodiments of this invention include the use of external magnetic fields that act upon magnetically labeled cells on the surface of an implant for purposes of producing mechanical stimulation of the cells, which may useful for triggering a specific response (e.g., secretion of mechanically-sensitive factors from cells or cell alignment) or for tracking the status of the labeled cell layer following implantation and monitoring cell proliferation.

While the present invention has been illustrated by the description of exemplary embodiments thereof, and while the embodiments have been described in certain detail, it is not the intention of the Applicant to restrict or in any way limit the scope of the appended claims to such detail. Additional advantages and modifications will readily appear to those skilled in the art. Therefore, the invention in its broader aspects is not limited to any of the specific details, representative devices and methods, and/or illustrative examples shown and described. Accordingly, departures may be made from such details without departing from the spirit or scope of the applicant's general inventive concept.

What is claimed:

1. A method for providing biocompatibility between an implant device and a biological target region, comprising:
(a) constructing an implant, wherein the implant further comprises:
(i) at least two surfaces; and
(ii) at least one magnet in communication with one of the surfaces;
(b) seeding the implant with magnetically labeled cells, wherein the cells are compatible with the target region, and wherein the magnetic attraction between the magnet and the magnetically labeled cells attaches the cells to the surface opposite the magnet; and
(c) implanting the implant in a target region, wherein cells proliferate to substantially cover the implant.

2. The method of claim 1, further comprising the step of adding at least one of a cell growth stimulating factor and a cell differentiation factor to the implant prior to implantation within the target region, wherein the implant device is a controlled release device, and wherein the cell growth stimulating factor or cell differentiation factor is released into the target region from the controlled release device.

3. The method of claim 1, wherein the biological target region further comprises cardiovascular tissue or bone.

4. The method of claim 1, wherein the implant is at least one of drug delivery device, pacemaker, stent, biosensor, orthopedic device, and medical device.

5. The method of claim 1, wherein the at least two surfaces further comprise plastic, silicon, titanium, or a combination thereof.

6. The method of claim 1, wherein the magnetically labeled cells further comprise stem cells, progenitor cells, mature cells, or a combination thereof.

7. A method for increasing the bioavailability of a composition, comprising:
(a) placing a biologically active agent in or on an implant, wherein the implant further comprises:
(i) a substrate having multiple surfaces; and
(ii) at least one magnet in communication with at least one surface of the substrate;
(iii) a means for releasing the biologically active agent; and
(b) seeding the implant with magnetically labeled precursor cells, wherein the cells are compatible with the target region, and wherein the magnetic attraction between the magnet and the magnetically labeled precursors cells attaches the cells to the surface of the substrate opposite the magnet; and
(c) implanting the implant in a biological target region, wherein the precursor cells differentiate and proliferate to substantially vascularize the target region around the implant, and wherein the biologically active agent is released from the implant and enters the vasculature.

8. The method of claim 7, further comprising the step of adding at least one of a cell growth stimulating factor and a cell differentiation factor to the implant prior to implantation within the target region, wherein the implant is a controlled release device, and wherein the cell growth stimulating factor or cell differentiation factor is released into the target region from the controlled release device.

9. The method of claim 7, wherein the biological target region further comprises cardiovascular tissue or bone marrow.

10. The method of claim 7, wherein the implant is at least one of a drug delivery device, pacemaker, stent, biosensor, orthopedic device, and medical device.

11. The method of claim 7, wherein the substrate further comprises at least one contoured surface.

12. The method of claim 7, wherein the housing further comprises plastic, silicon, titanium, or a combination thereof.

13. The method of claim 7, wherein the magnet in communication with the interior of the housing, the exterior of the housing, or is embedded in the material of the housing.

14. The method of claim 7, wherein the magnetically labeled precursor cells further comprise stem cells, progenitor cells, mature cells, or a combination thereof.
